(19) United States
(12) Patent Application Publication
(10) Pub. No.: US 2016/0008581 A1
(43) Pub. Date: Jan. 14, 2016

(54) NEEDLE SAFETY DEVICES AND RELATED METHODS

(71) Applicant: B. Braun Melsungen AG, Melsungen (DE)

(72) Inventors: Woon Chau Ang, Penang (MY); Soo Yong Tan, Penang (MY); Chee Mun Phang, Penang (MY)

(73) Assignee: B. Braun Melsungen AG, Melsungen (DE)

(21) Appl. No.: 14/793,040

(22) Filed: Jul. 7, 2015

Related U.S. Application Data
(60) Provisional application No. 62/021,870, filed on Jul. 8, 2014.

(51) Int. Cl. A61M 25/06

(52) U.S. Cl. CPC A61M 25/0618; A61M 2207/00

(57) ABSTRACT

Safety needle assemblies, needle guards, and related methods are disclosed. The guards may be used on different needle applications to cover the tips of the needles. Exemplary needle guards are disclosed in which a curved bore profile is provided so that when the crimp or bulge on the needle retracts into the curved bore profile, the axial movement of the needle causes the needle guard to undergo a rotary movement.

### FIELD OF ART

The present disclosure is generally directed to needle safety devices and more specifically to needle guards that cover needle tips following activation to prevent inadvertent needle sticks.

### BACKGROUND

Insertion procedure for an IV catheter assembly contains four basic steps: (1) the healthcare worker inserts the needle and catheter together into the patient's vein; (2) after insertion into the vein with the needle point, the catheter is forwarded into the vein of the patient by the healthcare worker pushing the catheter with his or her finger; (3) the healthcare worker withdraws the needle by grasping the hub end (opposite the point end) while at the same time applying pressure to the patient's skin at the insertion site with his or her free hand to stop the flow of blood through the catheter; and (4) the healthcare worker then tapes the exposed end of the catheter (the catheter hub) to the patient's skin and connects it to the source of the fluid to be administered into the patient's vein.

The problem is that, immediately after the withdrawal of the needle from the patient's vein, the healthcare worker, who is at this time involved in at least two urgent procedures, must place the exposed needle tip at a nearby location and address the tasks required to accomplish the needle withdrawal. It is at this juncture that the exposed needle tip creates a danger of an accidental needle stick, which, under the circumstances, leaves the healthcare worker vulnerable to the transmission of various dangerous blood-borne pathogens, including AIDS and hepatitis.

Other needle types similarly expose healthcare workers to risks of accidental needle sticks. For example, a doctor administering an injection, using a straight needle, a Huber needle, an epidural needle, etc., may place the used needle on a tray for subsequent disposal by a nurse. For the period between placing the used needle on a tray or a work station to the time it is discarded, the used needle is a potential source for disease transmissions for those that work near or around the needle.

Accordingly, all needles should be covered immediately following use to ensure greater worker safety. Ideally, the procedure for covering the needle tip should be passive, self-activating, or at least simple to perform. In addition, the device for covering the needle should be reliable and robust.

### SUMMARY

Needle guards that can rotate relative to a needle are disclosed. An exemplary needle guard, in moving from a proximal position on a needle to a distal position on the needle to shield the needle tip, can rotate as it moves to the distal position. The exemplary needle guard can have a shroud and a base. The shroud can rotate relative to the base. In another example, both the shroud and the base rotate relative to the needle.

A safety needle assembly is disclosed. The assembly can comprise: a needle guard comprising a shroud having two arms and a drum having a bore; wherein at least one of said shroud and said bore comprises a curved profile; a needle comprising a needle shaft extending through the bore of the drum and in between the two arms; said needle shaft comprising a needle tip and a change in profile located proximally of the needle tip; and wherein the needle guard is rotatable when the change in profile is pulled through the curved profile.

The safety needle assembly wherein the shroud can have a path and wherein the path can be offset from the bore.

The safety needle assembly can further comprise a catheter hub with a cavity and wherein the needle guard can be located in the cavity of the catheter hub.

The safety needle assembly wherein the drum can have a curved profile and a secure bore located proximally of the curved profile.

The safety needle assembly wherein the two arms each can have a distal end and wherein the distal end can have a slanted surface.

The safety needle assembly wherein the shroud can be rotatable relative to the drum.

The safety needle assembly wherein the shroud can have a split that separates the two arms and a continuous portion proximal of the split.

The safety needle assembly can further comprise a locking slot comprising an opening formed at a proximal section of the split.

The safety needle assembly can further comprise a first path through the drum and a second path through the shroud and wherein the first path is offset from the second path.

The safety needle assembly wherein the split can define a gap and wherein the gap can be wider at a distal portion of the shroud than a proximal portion of the shroud.

The safety needle assembly wherein each arm can include a track and wherein the track can have a curved surface.

Another aspect of the present disclosure is a method of making a safety needle assembly. The method can comprise: providing a needle comprising a needle shaft with a needle tip and a change in profile located proximally of the needle tip; forming a needle guard comprising a shroud having two arms and a drum having a bore; wherein at least one of said shroud and said bore comprises a curved profile; locating the needle shaft through the bore and in between the two arms; and causing the needle guard to rotate about the needle shaft when passing the change in profile through the curved profile.

The method can further comprise placing the needle guard into a cavity of a catheter hub.

The method wherein each of the two arms can include a projection formed on an exterior surface thereof.

The method wherein only the shroud can rotate.

The method wherein both the shroud and the drum can rotate.

The method, wherein the drum can have a first path and the shroud can have a second path and wherein the first path and the second path can be offset from one another.

The method can further comprise a projection formed on each of the two arms.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a schematic perspective view of a safety needle assembly provided in accordance with aspects of the present disclosure.

FIG. 2 is a schematic perspective view of the needle guard of FIG. 2, shown without a needle.

FIG. 3 is a side view of the needle guard of FIG. 2 in a protective position.

FIG. 4 is a schematic side view of a safety catheter assembly having the needle guard of FIG. 1 disposed inside the catheter hub.

FIG. 5 is a schematic partial transparent view of an alternative needle guard provided in accordance with aspects of the present disclosure.

FIG. 6 is a schematic perspective view of the needle guard of FIG. 5 in a protective position and shielding a needle tip.

### DETAILED DESCRIPTION

The detailed description set forth below in connection with the appended drawings is intended as a description of the presently preferred embodiments of needle safety devices, systems and related methods provided in accordance with aspects of the present disclosure and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

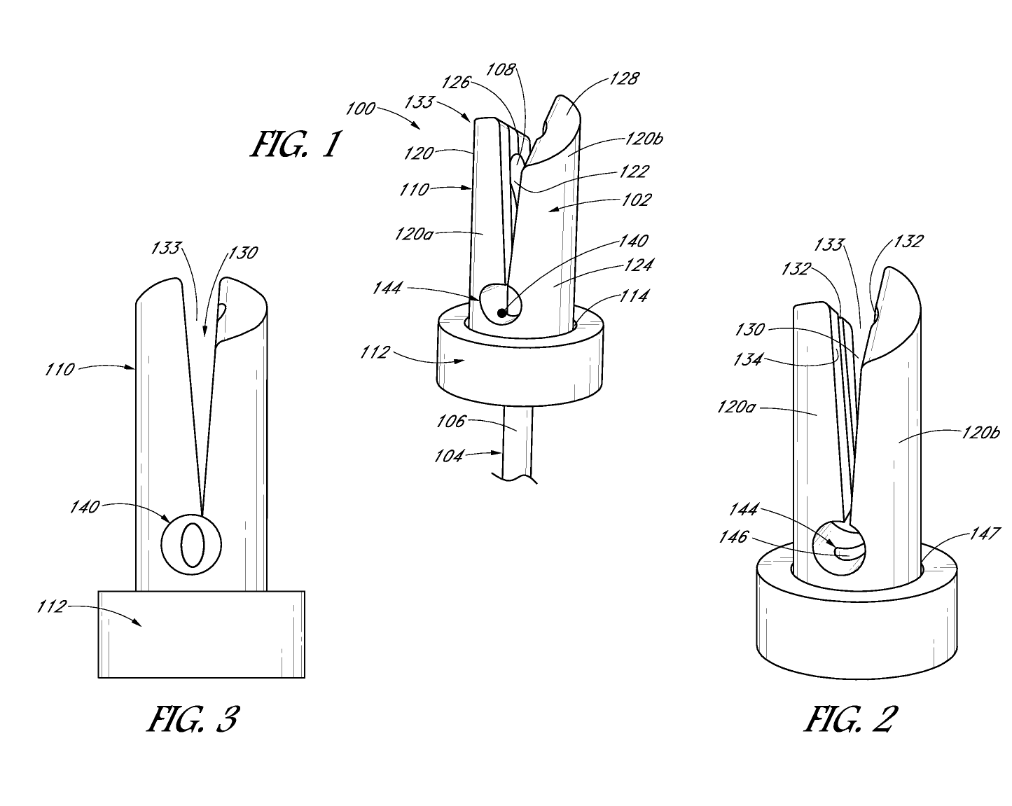

FIG. 1 illustrates a schematic perspective view of a needle safety device 100 comprising a needle guard 102 mounted on a needle 104 comprising a needle shaft 106 and a needle tip 108. The needle guard 102 may alternatively be referred to as a needle shield, a tip protector, or a needle cover. As shown, the needle guard 102 comprises a shroud 110 and a drum 112. In an example, the shroud 110 is connected to the drum 112. In another example, the shroud 110 is connected to the drum 112 and is movable relative to the drum, such as being rotatable relative to the drum. For example, the base or lower end of the shroud 110 may have one of a flange or a groove for rotating in the other one of a flange or a groove formed inside the bore 114 of the drum 112. The needle guard 102 may be formed by plastic molded injection of the shroud and the drum separately and then snap fitting the two together. In another example, the drum 112 may be formed from multiple pieces to facilitate assembly of the shroud into the bore of the drum. The separate pieces can be glued, welded, threaded, or snap fit together after installation using detents or the likes.

As shown, the shroud 110 comprises an elongated body 120 having a split 122 formed along a substantial portion of the body, such as from about 55% to about 95% of the length of the body to define an open cavity 126. The split is form across two opposed remote exterior surfaces of the body so that the body has at least two gaps or spaces that open into a center of the body. The shroud 110 further comprises a continuous body portion 124 without the split and below or proximal of the split near where the shroud 110 attaches to the drum 112. As used herein, the distal end is an end of the needle guard or needle safety device that is closer to the patient and proximal end is the end remote from the distal end.

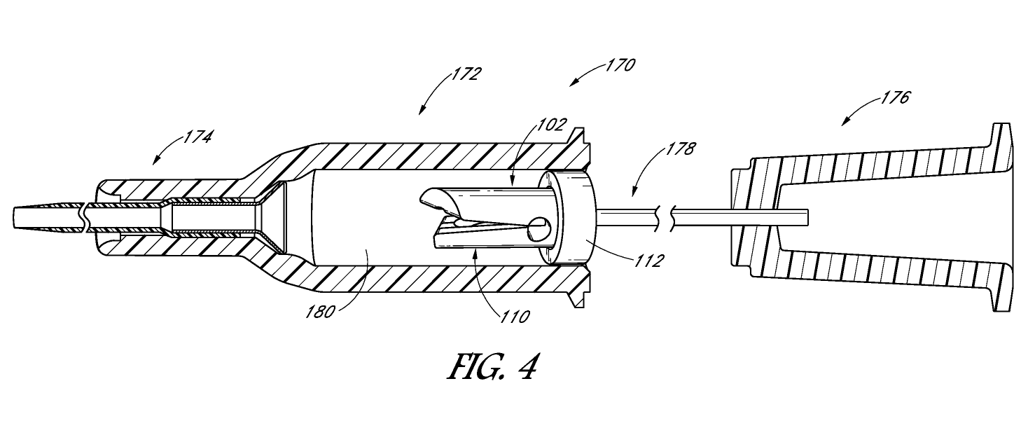

The split 122 defines two body sections 120a, 120b each with a slanted end 128, slanted relative to the planar distal surface of the drum 112. In other examples, the ends or at least one of the ends of the two body sections 120a, 120b are generally orthogonal to the lengthwise axis of the needle guard. Internally of the open cavity 126, a curved horn feature 130 in the form of a path or guide is provided. In one example, the curved horn feature 130 comprises a track 132 in the form of a channel formed on each of the interior surfaces 134 of the two split body sections 120a, 120b. The two tracks 132 or channels together define a needle path 133. Each channel 132 (FIG. 2) resembles a partial curve or arc section of a circle or an oval and is sized and shaped to guide the needle shaft 106 and the needle crimp or bulge located proximally of the needle tip 108. When the needle 104 is in a ready to use position (not shown) with the needle tip 108 extending distally of the needle guard 102, the girth of the needle slightly separates the two body sections 120a, 120b. In other words, the gap or space of the open cavity 126 near the continuous portion 124 is smaller than the needle diameter. In a different example, the split 122 is sized so that the needle 104 touches but does not expand the two body sections apart. In still another example, the needle is spaced from and does not touch the two body sections 120a, 120b. As used herein, a crimp may be formed by compressing the needle shaft to form a deformity having both a recessed section and an enlarged section. A bulge may be an enlarged section of a crimp or formed separately, such as from a material buildup or an add-on piece, such as a sleeve. As used herein, the crimp and bulge may more generically be referred to as a change in profile, which is a change in a nominal shaft diameter of the needle shaft.

The two channels 132 are generally parallel to one another along the split 122 but can converge in the proximal direction. The two channels each has a break or terminates at a slot 140, which is formed as a through hole in the elongated body 120 at a location above the drum 112, above the continuous portion 124 of the elongated body 120, or both. In one example, the through hole 140 is generally circular with the center of the circle being off-set from an axis or path defined by the two channels 132, which is more clearly shown in FIGS. 2 and 3. The off-set configuration of the through hole 140 is configured to trap the change in profile formed with the needle to prevent movement of the needle relative to the guard following activation of the needle guard to cover the needle tip. The through hole or slot 140 may be referred to as a locking slot or catch. In another example, the outside surfaces of the two body sections 120a, 120b of the shroud are generally constant along a radial direction, such as having similar outside diameters along a distal portion and a proximal portion, but the interior surfaces 134 of the two body sections both diverge or converge, depending on the direction the needle path 133 is viewed, and is skewed thereby forming a horn feature 130.

With reference now to FIG. 2, another path 144 is shown formed at the proximal end 147 of the shroud 110, at the continuous section 124 of the body 120. The path 144 has an opening 146 leading into a bore that extends the remaining portion of the shroud 110. For discussion purposes, the path 144 in the proximal end of the shroud may be referred to as a first path and the path 133 defined by the split section 122 as a second path. The opening 146 of the first path 144 is generally round but can be oval or elliptical. The first path 144 allows the needle shaft 106 to pass therethrough but not the change in profile, such as a crimp. In other words, when the needle is retracted in the proximal direction to move the needle tip 108 of the needle inside the shroud 110 to be covered by the shroud, as shown in FIG. 1, the crimp or bulge on the needle is too large to move proximally of the opening 146 of the first path 144, or proximally any substantial amount of the opening 146. Said differently, the diameter of the opening 146 is smaller than the largest cross-sectional dimension of the change in profile so that the change in profile cannot materially pull through the opening. This arrangement is configured to limit the needle from moving too far in the proximal direction to separate from the needle guard 102.

With reference now to FIGS. 1 and 3, upon retracting the needle 104 and the needle tip 108 inside the shroud 110 to a point in which the change in profile on the needle, such as a crimp, reaches the catch or locking slot 140, the off-set configuration causes the portion of the needle distal of the change in profile, such as a crimp or bulge, to align with the track 132 while the needle shaft proximal of the crimp or bulge aligns with the first path 144. As the first path 144 and the track 132 are offset, the needle will be wedged by different sections of the needle guard 102 to retain the needle guard over the needle tip. For the needle to move away from this secured position at the catch 140, this would require a relatively significant force in order to move the needle away from the off-set configuration, which is difficult if not impossible. Once the change in profile of the needle is in the locking slot 140, this will prevent the needle tip from re-emerging out the needle guard 102 as the track 132 diameter is smaller than the change in profile. Further, the offset configuration causes a bending moment on the needle that presses against the inside of the track, which makes sliding the needle relative to the needle guard difficult following securement of the needle guard over the needle tip. The change in profile, such as a crimp, will be stopped by the smaller dimension of the track 132 and the bending moment, hence preventing the needle tip from re-emerging from the needle guard.

In one example, the needle guard 102 may be used on any needle having change in profile, such as a crimp or a bulge. For example, a change in profile can be used with a Seldinger needle, a Touhey needle, or a straight injection needle. The needle guard 102 can be located on a proximal position on the needle spaced from the needle tip and can then manually moved by a user to a distal position on the needle to cover the needle tip following use.

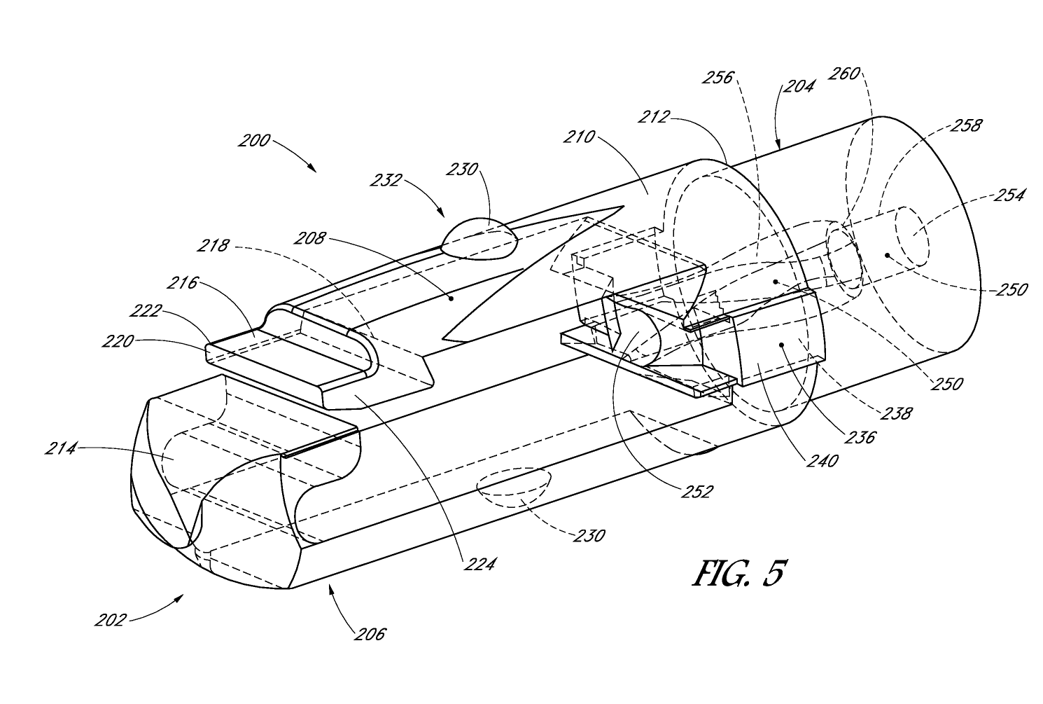

In another example, the needle guard 102 is used with a catheter assembly. With reference now to FIG. 4, a schematic side view of an over the needle catheter assembly 170 is shown, which comprises a catheter hub 172 with a catheter tube 174 and a needle hub 176 with a needle 178 comprising a change in profile, such as a crimp or a bulge, near the needle tip. The assembly 170 is shown with a needle guard 102 disposed in an interior cavity 180 of the catheter hub 172, which can be the same as the needle guard 102 discussed above with reference to FIGS. 1-3. The catheter hub 172 has a proximal opening with a Luer taper. The needle guard 102 is shown positioned in the Luer taper with the drum 112 frictionally engaged with the interior surface of the catheter hub. In some examples, the drum has a slight interference fit with the Luer taper. The nose of the needle hub 176 can project slightly into the catheter hub when in a ready to use position with the needle tip extending distally of the distal opening of the catheter tube. In other examples, none of the needle hub projects into the catheter hub but a fixed position can be established between the needle hub and exterior surfaces of the catheter hub. Following use, the needle 178 is retracted in the proximal direction until the change in profile, such as a crimp, on the needle is trapped at the locking slot 140 of the needle guard 102. At that point, further proximal movement of the needle causes the crimp to pull on the needle guard 102 in the proximal direction to separate the needle guard from the catheter hub.

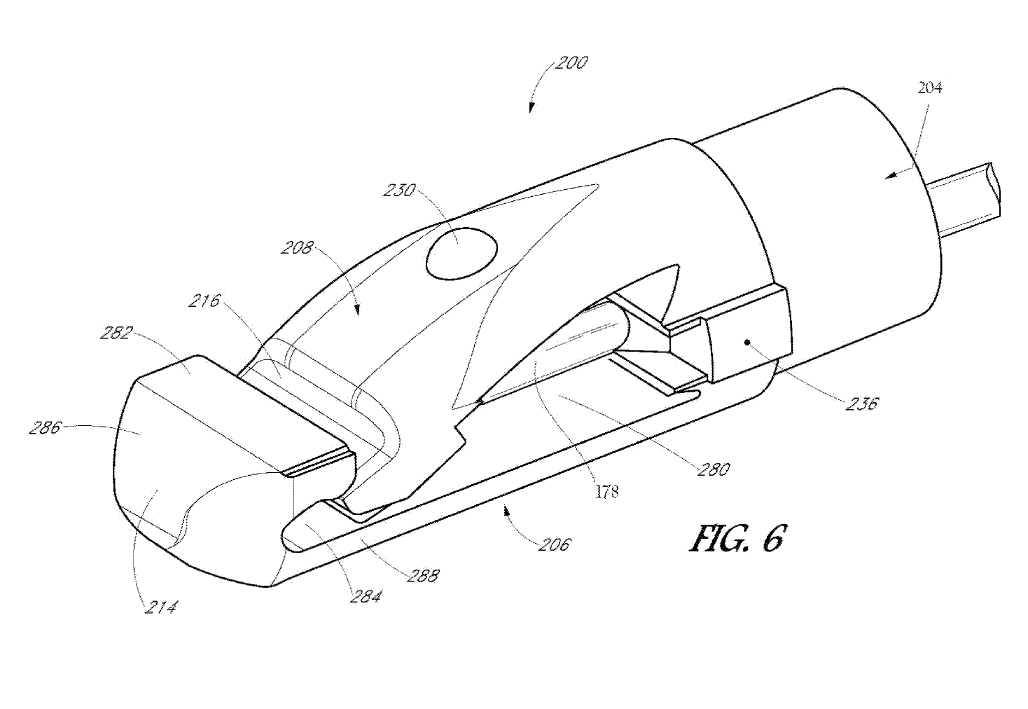

FIG. 5 is a schematic perspective semi-transparent view of an alternative needle guard 200 provided in accordance with further aspects of the present disclosure. The present needle guard 200 may be used with an injection needle or as part of a catheter assembly, similar to the manner in which the needle guard 102 of FIG. 4 is used with a catheter assembly. The needle guard 200 of the present embodiment, which may also be referred to as a tip protector, a needle shield, or a needle cover, may be made by plastic molded injection as a one-piece device or structure. In an example, the needle guard 200 comprises a shroud 202 and a drum 204. As shown, the drum 204 is generally cylindrical and the shroud 202 comprises two arms 206, 208 that extend distally of the drum. In one example, the shroud has a continuous base 210 that extends from the drum 204 with a shoulder 212 provided therebetween. The two arms, i.e., the first arm 206 and the second arm 208, extend from the base 210 with the first arm 206 extending longer than the second arm 208 and the first arm has an overhang 214 that functions as a blocking distal wall for blocking a needle tip following use when the needle guard 200 is used with a needle. The second arm 208 has a shaped distal end portion 216 comprising a tapered or reduced joint 218 and radiused corners 220, 222, 224. The shaped distal end portion 216 is provided to project below the overhang 214 on the first arm 206, as further discussed below. The arms can be reversed so that the overhang is with the second arm and the first arm has a shaped distal end.

A projection 230 is provided on an exterior of each arm 206, 208, which can embody a partial sphere. The two projections 230 on the exterior of each arm may be referred to as a first projection set 232 and are configured to cooperate with a second projection set 236. The second projection set 236 comprises a projection 238 formed at a spaced apart distance from at least one other projection 238 on the base 210 of the shroud 202. As shown, there are a total of four projections 230, 230, 238, 238 that are equally spaced around the guard along a radial direction. In other examples, there may be fewer or more than four projections formed on the exterior surfaces of the guard 200 with the present guard comprising four projections formed on the shroud 202. The projections 238 on the base 210 are generally rectangular with the surface 240 of the projections being arcuate, such as being generally curved to resemble the curvature of the interior surface of the hub.

As further discussed below, the needle guard 202 may be used with an over-the-needle catheter or on other needle types, such as a straight needle. When used with an over-the-needle catheter, the guard is placed inside a hub, such as inside a catheter hub or inside a guard housing located proximally of the catheter hub. The four projections 230, 230, 238, 238 are sized and shaped to guide the guard within the interior cavity of the hub. For example, the four projections steady the guard 200 within the interior cavity of the hub so that the guard can rotate about the lengthwise axis of the guard using the projections as balancing means for rotating, similar to bearings.

Interiorly, a bore 250 is provided with an opening 252 on the distal end and an opening 254 on the proximal end of the bore. The bore 250 has at least two bore sections, including a rotary profiled bore section 256 and a securement bore section 258. The distal opening 252 of the bore 250 is sized and shaped to receive a change in profile, such as a crimp or a bulge, on a needle. In the embodiment shown, the distal opening 252 is non-circular, such as having a perimeter that is oval or elliptical in shape. The non-circular opening 252 extends into the rotary profiled bore section 256 in a rotary manner. In other words, if the non-circular opening 252 has a major axis along the larger dimension of the opening and a minor axis along the smaller dimension of the opening, the position of the major axis, and hence the minor axis, rotates in a clockwise manner as it extends towards the securement bore section 258. The rotary profiled bore section 256 therefore has a helical shape. In other examples, the helical shape has a counter-clockwise rotation. A transition bore section 260 is located between the rotary profiled bore section 256 and the securement bore section 258, which in the present embodiment is generally cylindrical, such as having a generally cylindrical bore. The securement bore section 258 has a bore diameter that is smaller than the largest dimension of a change in profile, such as a crimp or bulge, employed on the needle that the guard 200 is to be used with. This allows the securement bore section 258 to engage the change in profile on the needle to stop the needle from being dislodged, such as to stop movement of the needle relative to the guard in the proximal and distal directions relative to the needle guard 200.

The needle guard 200 may be used with a straight needle or with an over-the needle catheter application, as previously mentioned. For an over-the-needle application, such as an IV catheter application, the guard is placed inside a hub, such as a catheter hub, with the first projection set 230 and the second projection set 236 touching or nearly touching the interior surface of the catheter hub. Further, a retention feature R (not shown), such as annular projection, can be provided on the interior surface of the hub and the two projections 238 on the base 210 of the guard are advanced just distal of the retention feature R in order to secure the guard inside the hub and using the retention feature R to prevent the guard from prematurely dislodging from the hub in the proximal direction. The retention feature R is configured to retain the needle guard 200 during use and during transition of the needle from a ready to use position to a protective position following an injection in which the needle tip is moved inside the needle guard 200. In other words, the retention feature R is provided to prevent premature separation of the needle guard 200 from the hub during movement of the needle from the ready position to the retracted position. Thus, in an exemplary embodiment, the drum 204 of the needle guard 200 does not contact the interior surface of the catheter hub.

In the ready position, the needle projects through the guard 200 and between the two arms 206, 208. In other words, the presence of the needle urges the two arms 206, 208 radially outwardly. As the needle is retracted into the guard 200 so that the guard covers the needle tip, the crimp or bulge on the needle enters the rotary profiled bore section 256. As that section of the bore is helical or spiral, the crimp or bulge on the needle causes the guard to rotate about the rotary profiled bore section 256. This is analogous to pulling a screw through a nut and allowing the nut to freely rotate.

As the crimp or bulge reaches the transition section 260 of the bore 250, the change in profile on the needle, such as a crimp or bulge, is prevented from further displacing into the securement bore section 258, or at least any substantial amount of travel into the securement bore section 258 due to the relative sizes of the two components. In an example, the change in profile engages the securement bore section 258 in an interference fit with the securement bore section 258 having a bore diameter that is smaller than the largest outer dimension of the change in profile. For a straight needle application, the guard 200 is now positioned over the needle tip and prevents inadvertent needle sticks with the needle tip. For an IV catheter application, further retraction of the needle in the proximal direction will cause the second projection set 236 to retract away from the retention feature R inside the catheter hub. The guard and the needle can then completely separate from the hub.

With reference now to FIG. 6, the guard is shown in an activated position over a needle 178 and shielding the tip of the needle from un-intended needle sticks. The tip is retracted so that the needle tip is in the open cavity 280 area of the guard 200. As shown, both arms 206, 208 of the needle guard collapse radially inwardly when no longer held opened by the needle 178. In another example, only the second arm 208 moves radially inwardly but not the first arm 206 or only the first arm moves radially inwardly but not the second arm. As shown, the shaped end 216 of the second arm 208 moves below the end bar 282 on the overhang section 214 of the first arm. This is configured so that if attempt is made to re-expose the needle 178, the second arm 208 pushes the needle tip into the distal cavity section 284 defined by the end bar 282, the distal wall 286, and the elongated arm portion 288 of the first arm 206.

Method of making and using the needle guards described herein with various number of needle devices are understood to be within the scope of the present disclosure.

Although limited embodiments of safety needle devices and needle guards and related methods have been specifically described and illustrated herein, many modifications and variations will be apparent to those skilled in the art. Accordingly, it is to be understood that safety needle devices and needle guards and related methods and their components constructed according to principles of the disclosed devices, systems, and methods may be embodied other than as specifically described herein. The disclosure is also defined in the following claims.

What is claimed is:

1. A safety needle assembly comprising:
a needle guard comprising a shroud having two arms and a drum having a bore; wherein at least one of said shroud and said bore comprises a curved profile;
a needle comprising a needle shaft extending through the bore of the drum and in between the two arms; said needle shaft comprising a needle tip and a change in profile located proximally of the needle tip; and
wherein the needle guard is rotatable when the change in profile is pulled through the curved profile.

2. The safety needle assembly of claim 1, wherein the shroud has a path and wherein the path is offset from the bore.

3. The safety needle assembly of claim 1, further comprising a catheter hub with a cavity and wherein the needle guard is located in the cavity of the catheter hub.

4. The safety needle assembly of claim 1, wherein the drum has a curved profile and a secure bore located proximally of the curved profile.

5. The safety needle assembly of claim 1, wherein the two arms each has a distal end and wherein the distal end has a slanted surface.

6. The safety needle assembly of claim 1, wherein the shroud is rotatable relative to the drum.

7. The safety needle assembly of claim 1, wherein the shroud has a split that separates the two arms and a continuous portion proximal of the split.

8. The safety needle assembly of claim 7, further comprising a locking slot comprising an opening formed at a proximal section of the split.

9. The safety needle assembly of claim 1, further comprising a first path through the drum and a second path through the shroud and wherein the first path is offset from the second path.

10. The safety needle assembly of claim 7, wherein the split defines a gap and wherein the gap is wider at a distal portion of the shroud than a proximal portion of the shroud.

11. The safety needle assembly of claim 1, wherein each arm has a track and wherein the track has a curved surface.

12. A method of making a safety needle assembly comprising:
providing a needle comprising a needle shaft with a needle tip and a change in profile located proximally of the needle tip;
forming a needle guard comprising a shroud having two arms and a drum having a bore; wherein at least one of said shroud and said bore comprises a curved profile;
locating the needle shaft through the bore and in between the two arms; and
causing the needle guard to rotate about the needle shaft when passing the change in profile through the curved profile.

13. The method of claim 12, further comprising placing the needle guard into a cavity of a catheter hub.

14. The method of claim 13, wherein each of the two arms has a projection formed on an exterior surface thereof.

15. The method of claim 12, wherein only the shroud rotates.

16. The method of claim 12, wherein both the shroud and the drum rotate.

17. The method of claim 12, wherein the drum has a first path and the shroud has a second path and wherein the first path and the second path are offset from one another.

18. The method of claim 12, further comprising a projection formed on each of the two arms.
